(19) United States
(12) Patent Application Publication
(10) Pub. No.: US 2022/0381791 A1
(43) Pub. Date: Dec. 1, 2022

(54) METHOD FOR DETERMINING AN INDIVIDUAL ABILITY TO RESPOND TO A STIMULUS

(71) Applicants: BIOMÉRIEUX, Marcy-l'Etoile (FR); HOSPICES CIVILS DE LYON, Lyon (FR); UNIVERSITE CLAUDE BERNARD LYON 1, Villeurbanne (FR)

(72) Inventors: Sophie ASSANT, Reyrieux (FR); François MALLET, Villeurbanne (FR); François BARTOLO, Nice (FR); Chloé ALBERT VEGA, L'Eliana (ES)

(73) Assignees: BIOMÉRIEUX, Marcy-l'Etoile (FR); HOSPICES CIVILS DE LYON, Lyon (FR); UNIVERSITE CLAUDE BERNARD LYON 1, Villeurbanne (FR)

(21) Appl. No.: 17/766,155

(22) PCT Filed: Sep. 30, 2020
(86) PCT No.: PCT/FR2020/051715

§ 371 Date: Apr. 1, 2022

(30) Foreign Application Priority Data
Oct. 1, 2019 (FR) 19/10884
Dec. 18, 2019 (EP) 19217548.7

(51) Int. Cl. G01N 33/68; C07K 16/28; C12Q 1/6813; C12Q 1/6844

(52) U.S. Cl. CPC G01N 33/6818; C07K 16/2809; C12Q 1/6813; C12Q 1/6844; C12Q 2600/158

(57) ABSTRACT

An in vitro or ex vivo method for determining the ability of an individual to respond to a stimulus, based on the measurement of the expression of at least two different biomarkers, selected from different lists among three lists of biomarkers, from a blood sample of the individual, incubated with the stimulus, as well as tools allowing the implementation of this method and the use of these tools.

The present invention concerns an in vitro or ex vivo method to determine the ability of an individual to respond to a stimulus, based on the measurement of the expression of at least two different biomarkers, selected from different lists among three lists of biomarkers, from a blood sample of said individual, incubated with said stimulus, as well as tools allowing the implementation of this method and the use of these tools.

The immune system is the body defense system against what is recognized as non-self, such as pathogens. The immune response requires very fine regulation and can sometimes be altered, in particular in the case of inflammatory, allergic or autoimmune diseases (in which the immune system is more active than normal), or diseases characterized by immunosuppression (in which the immune system is less active than normal). This immunosuppression can have different origins, take many forms, and affect innate immunity and/or adaptive immunity.

In particular, sepsis was recognized as a health priority by the WHO in 2017, and represents a global problem in terms of morbidity, mortality, as well as costs. It is estimated that 31.5 million people develop sepsis each year worldwide, of which 6 million will die from the disease and 3 million will suffer from disorders that can lead to readmission to hospital. In a patient with sepsis (also known as in a septic state), the immune response is dysregulated, following an infection, which leads to multiple and life-threatening organ failure and dysfunction. This immune response is complex and evolves over time, with excessive pro-inflammatory and anti-inflammatory phenomena that can be concomitant. All of these immune system disorders lead to organ failure, immune system paralysis and secondary infections. Septic shock is a subtype of sepsis, in which hypotension persists, despite adequate vascular filling. At the initial stage of sepsis, it is an inflammatory or even hyper-inflammatory response (including cytokine shock), which seems to predominate, and which is the cause of tissue damage and organic failures, particularly at the renal level. This is why clinical trials in the field of sepsis have long focused on anti-inflammatory treatments, but with inconclusive results. More recent studies on the pathophysiology of sepsis have shown that an anti-inflammatory or immunosuppressive response occurs in sepsis patients, either concomitant with the initial inflammation or later, in an attempt to offset the hyper-inflammatory response. The patient can then find himself in a state of immunosuppression, potentially severe, depending on the respective degrees of pro-inflammatory and anti-inflammatory responses. These immunocompromised patients are at high risk of developing nosocomial infections (or HAI, Hospital-Acquired Infections or Healthcare-Associated Infections) and of being prone to viral reactivation, and could advantageously benefit from immunostimulant treatments. However, early studies in patients with septic shock showed no benefit with such treatments. This may be due to the complexity of the pathophysiology of sepsis (including inter-individual variability in the immune response), but also to the dynamics of the host response.

The stratification of patients according to their immunological profile therefore seems essential to their effective management. A diagnostic tool allowing precise identification of the functionality of the immune system and the immune status is of fundamental importance, in order to be able to adapt and personalize the therapeutic management. Yet, individuals with immune system disorders do not show specific clinical signs; in particular, the interpretation of the host response in septic patients remains a challenge. Soluble or membrane biomarkers have been proposed, such as the expression of HLA-DR (human leukocyte antigen-D related) on the surface of monocytes (mHLA-DR) or the expression of CD88 in neutrophils, as well as the count lymphocytes or platelets, but they are each restricted to a single cell population, which probably underestimates the overall immune contribution.

In certain clinical situations (such as latent tuberculosis), functional tests, or immune functional tests (IFA, Immune Functional Assays), have made it possible to significantly improve patient care. Functional tests measure directly, ex vivo, the ability of one or more cell population(s) to respond to a stimulus with which the cells are brought into contact, and have for example been used in research to study the energy of monocytes, most often by measuring TNFα at the protein level after ex vivo stimulation with lipopolysaccharide (LPS), as well as clinically, in the case of tuberculosis, by measuring interferon γ at the protein level after stimulation with a Mycobacteria tuberculosis antigen. Functional tests were also used as part of a study aimed at defining the limits of a normal immune response (i.e. in a «healthy» context) in response to different infectious challenges (Urrutia et al (2016), Cell Reports 16: 2777-2791).

Yet, it has been discovered that, quite surprisingly, functional tests based on measuring the expression of certain specific biomarkers, classified into three lists, from an individual blood sample, incubated with a stimulus, made it possible to determine the capacity of this individual (which could be either a healthy individual or a sick individual, such as a patient suffering from sepsis) to respond to this stimulus. In particular, these functional tests make it possible to highlight the inter-patient heterogeneity of the immune response, dynamically, in terms of dysfunctions of the innate and/or adaptive immune response, and therefore to capture the singularity of the ability to response of each patient, so as to deduce useful information about the diagnosis, prognosis and/or therapeutic management of the patient. The functional test according to the invention makes it possible in particular to highlight three categories of individuals: individuals presenting an unaltered to slightly altered immune profile (cluster S1), individuals presenting a strongly altered immune profile (cluster S2) and individuals with an intermediate immune profile (cluster S3). Individuals in cluster S2, whose immunity appears to be greatly altered and presenting a greater probability of mortality, could advantageously benefit from more «aggressive» and/or earlier therapeutic interventions, while the standard of care would be sufficient for individuals of the cluster S1, whose immunity is little altered; in individuals of the cluster S3, whose immunity appears to be restorable, personalized treatments (e.g. IL-7, interferonγ) could advantageously be tested.

Thus, the present invention relates to an in vitro or ex vivo method for determining the ability of an individual to respond to a stimulus, preferably to determine the ability of an individual immune system to respond to a stimulus, comprising:

a) A step of incubating a blood sample of said individual with said stimulus, and

b) A step of measuring the expression, from the stimulated blood sample resulting from step a), of at least two different biomarkers, selected respectively from at least two different lists, from the following lists:

List S1: BST2, CCL20, CCL4, CCL8, CD209, CD3D, CD44, CD74, CD83, CLEC7A, CXCL10, CXCL2, CXCL9, DYRK2, FAM89A, HLA-DMB, HLA-DPB1, IFNG, IL1A, IRAK2, PTGS2, RARRES3, DDX58, SLAMF7, SRC, STAT2,STING, TNFA, TNFSF13B, ZBP1;

List S2: ADGRE3, ARL14EP, BST2, C3, CCL2, CCL20, CCL8, CCNB1IP1, IL7R, CD209, CD3D, CD44, CD74, CD83, CDKN1A, CLEC7A, CX3CR1, CXCL10, CXCL2, CXCL9, DYRK2, FAM89A, HLA-DMB, HLA-DPB1, HLA-DRA, IFITM1, IRAK2, SLAMF7, TGFB1;

List S3: 121601901-HERV0116, BST2, C3, CCL20, CCL4, CCL8, CCR1, IL7R, CD209, CD44, CD74, CD83, CLEC7A, CXCL10, CXCL9, EIF2AK4, HLA-DMB, HLA-DPA1, HLA-DPB1, HLA-DRA, IL1A, IL2, RARRES3, SLAMF7, STAT2.

TABLE 1
Chromosomal location of biomarkers according to GRCh38/hg38

| Target biomarkers | Chromosomal location (GRCh38/hg38) |
|---|---|
| ADGRE3 | chr19: 14, 619, 117-14, 690, 027 |
| ARL14EP | chr11: 30, 323, 099-30, 338, 458 |
| BST2 | chr19: 17, 402, 939-17, 405, 648 |
| C3 | chr19: 6, 677, 704-6, 730, 562 |
| CCL2 | chr17: 34, 255, 218-34, 257, 203 |
| CCL20 | chr2: 227, 805, 739-227, 817, 564 |
| CCL4 | chr17: 36, 103, 827-36, 105, 621 |
| CCL8 | chr17: 34, 319, 047-34, 321, 402 |
| CCNB1IP1 | chr14: 20, 311, 368-20, 333, 312 |
| CCR1 | chr3: 46, 201, 709-46, 208, 341 |
| CD209 | chr19: 7, 739, 988-7, 747, 605 |
| CD3D | chr11: 118, 338, 954-118, 342, 744 |
| CD44 | chr11: 35, 138, 870-35, 232, 402 |
| CD74 | chr5: 150, 400, 041 -150, 412, 936 |
| CD83 | chr6: 14, 117, 256-14, 136, 918 |
| CDKN1A | chr6: 36, 676, 460-36, 687, 339 |
| CLEC7A | chr12: 10, 116, 777-10, 130, 273 |
| CX3CR1 | chr3: 39, 263, 494-39, 281, 735 |
| CXCL10 | chr4: 76, 021, 116-76, 023, 536 |
| CXCL2 | chr4: 74, 097, 035-74, 099, 280 |
| CXCL9 | chr4: 76, 001, 275-76, 007, 523 |
| DDX58 | chr9: 32, 455, 302-32, 526, 324 |
| DYRK2 | chr12: 67, 648, 338-67, 665, 406 |
| EIF2AK4 | chr15: 39, 934, 115-40, 035, 596 |
| FAM89A | chr1: 231, 018, 958-231, 040, 254 |
| HLA-DMB | chr6: 32, 934, 629-32, 941, 070 |
| HLA-DPA1 | chr6: 33, 064, 569-33, 080, 778 |
| HLA-DPB1 | chr6: 33, 075, 926-33, 089, 696 |
| HLA-DRA | chr6: 32, 439, 842-32, 445, 046 |
| IFITM1 | chr11: 313, 506-315, 272 |
| IFNG | chr12: 68, 154, 768-68, 159, 741 |
| IL1A | chr2: 112, 773, 915-112, 785, 394 |
| IL2 | chr4: 122, 451, 470-122, 456, 725 |
| IL7R | chr5: 35, 852, 695-35, 879, 603 |
| IRAK2 | chr3: 10, 164, 879-10, 243, 745 |
| PTGS2 | chr1: 186, 671, 791 -186, 680, 427 |
| RARRES3 | chr11: 63, 536, 801 -63, 546, 462 |
| SLAMF7 | chr1: 160, 739, 057-160, 754, 821 |
| SRC | chr20: 37, 344, 685-37, 406, 050 |
| STAT2 | chr12: 56, 341, 597-56, 360, 253 |
| STING1 | chr5: 139, 475, 533-139, 482, 758 |
| TGFB1 | chr19: 41, 301, 587-41, 353, 933 |
| TNFA | chr6: 31, 575, 565-31, 578, 336 |
| TNFSF13B | chr13: 108, 251, 240-108, 308, 484 |
| ZBP1 | chr20: 57, 603, 846-57, 620, 576 |
| 121601901-HERV0116 | chr12: 112972627-112975754 |

In the context of the present invention:

The term «individual» designates a human being, whatever he is (and in particular whatever his state of health, whether he is a healthy individual or a sick individual). The term «patient» designates an individual who has come into contact with a health professional, such as a doctor (for example, a general practitioner) or a medical structure (for example, a hospital, and more particularly the emergency room, resuscitation unit, intensive care unit or continuing care unit). A patient is io generally a sick individual, but it can also be a healthy individual (for example, an elderly person coming to be vaccinated);

The «stimulus» corresponds to one or more molecules capable of inducing an immune response and allowing the qualitative and/or quantitative evaluation of the individual immune response; in particular, they may be immunogen(s) (or «challenge(s)») or molecule(s) for therapeutic purposes;

Determining the «capacity of an individual to respond to a stimulus» can have several uses, both diagnostic (e.g. identifying the immune status of the individual, which can be a normal status, an inflammation status or a immunosuppression) and prognostic (e.g. identifying individuals whose immune status may evolve—for example, from a normal status to an inflammatory status or vice versa, or even individuals who will go from an immunosuppression status to an inflammation status), in order for example to adapt the therapeutic management, or even to predict and/or monitor the effectiveness of response to a treatment;

A «blood sample» means a sample of whole blood or a cell sample derived from blood (i.e. a sample obtained from blood and containing at least one type of cell, such as a sample of peripheral blood mononuclear cells or PBMC);

A «biomarker» or «marker» is an objectively measurable biological characteristic that represents an indicator of normal or pathological biological processes or of pharmacological response to a therapeutic intervention. It may in particular be a molecular biomarker, preferably detectable at the mRNA level. More particularly, the biomarker can be an endogenous biomarker or loci (such as a gene or a HERV/Human Endogenous Retro Virus, which are found in the chromosomal material of an individual) or an exogenous biomarker (such as a virus);

Preferably, in the method as described above, the at least two different biomarkers are selected respectively from at least two different lists, among the following lists:

List S1-1: BST2, CCL20, CCL4, CCL8, CD209, CD3D, CD44, CD83, CXCL2, DYRK2, HLA-DMB, IFNG, IL1A, IRAK2, PTGS2, RARRES3, DDX58, SRC, STAT2, STING, TNFA, TNFSF13B, ZBP1;

List S2-1: ADGRE3, ARL14EP, C3, CCL2, CCNB1IP1, IL7R, CD3D, CD44, CDKN1A, CLEC7A, CX3CR1, CXCL2, DYRK2, HLA-DMB, HLA-DRA, IFITM1, IRAK2, TGFB1;

List S3-1: 121601901-HERV0116, C3, CCR1, IL7R, CD44, CD74, CXCL10, CXCL9, ElF2AK4, HLA-DMB, HLA-DPA1, HLA-DPB1, HLA-DRA, IL1A, IL2, RARRES3, SLAMF7, STAT2.

Preferably again, in the method as described above, the at least two different biomarkers are selected respectively from at least two different lists, among the following lists:

List S1-2: CCL20, CCL4, CCL8, CD209, CD44, CD83, CXCL2, IFNG, IL1A, IRAK2, PTGS2, DDX58, SRC, STING, TNFA, TNFSF13B, ZBP1;

List S 2-2: ADGRE3, ARL14EP, CCL2, CCNB1IP1, IL7R, CDKN1A, CLEC7A, CX3CR1, DYRK2, IFITM1, TGFB1;

List S3-2: 121601901-HERV0116, C3, CCR1, CXCL10, CXCL9, EIF2AK4, HLA-DMB, HLA-DPA1, IL2, SLAMF7.

Even more preferably, in the method as described above, the at least two different biomarkers are selected respectively from at least two different lists, among the following lists:

List S1-3: IFNG, PTGS2, DDX58, SRC, STING, TNFA, TNFSF13B, ZBP1;

List S2-3: ADGRE3, ARL14EP, CCL2, CCNB1IP1, CDKN1A, CX3CR1, IFITM1, TGFB1;

List S3-3: 121601901-HERV0116, CCR1, EIF2AK4, HLA-DPA1, IL2.

Preferably, the method as described above is an in vitro or ex vivo method for determining the ability of an individual to respond to a stimulus, preferably to determine the ability of an individual immune system to respond to a stimulus, comprising:

a) A step of incubating a blood sample of said individual with said stimulus, and

b) A step of measuring the expression, from the stimulated blood sample resulting from step a), of at least three different biomarkers, selected respectively from:

List S1, List S2 and List S3;

List S1-1, List S2-1 and List S3-1;

List S1-2, List S2-2 and List S3-2: or

List S1-3, List S2-3 and List S3-3.

Preferably again, in step b) above, the expression is measured, from the stimulated blood sample resulting from step a):

of at least 4, at least 5, at least 6, at least 7, at least 8, at least 9, at least 10, at least 11, at least 12, at least 13, at least 14, at least 15 , at least 16, at least 17, at least 18, at least 19, at least 20, at least 21, at least 22, at least 23, at least 24, at least 25, at least 26, at least 27, at least at least 28, at least 29, at least 30, at least 31, at least 32, at least 33, at least 34, at least 35, at least 36, at least 37, at least 38, at least 39, at least 40 , at least 41, at least 42, at least 43, at least 44, at least 45, at least 46 different biomarkers selected from each of Lists S1, S2 and S3;

of at least 4, at least 5, at least 6, at least 7, at least 8, at least 9, at least 10, at least 11, at least 12, at least 13, at least 14, at least 15 , at least 16, at least 17, at least 18, at least 19, at least 20, at least 21, at least 22, at least 23, at least 24, at least 25, at least 26, at least 27, at least at least 28, at least 29, at least 30, at least 31, at least 32, at least 33, at least 34, at least 35, at least 36, at least 37, at least 38, at least 39, at least 40, at least 41, at least 42, at least 43, at least 44, at least 45 different biomarkers selected from each of Lists S1-1, S2-1 and S3-1;

of at least 4, at least 5, at least 6, at least 7, at least 8, at least 9, at least 10, at least 11, at least 12, at least 13, at least 14, at least 15 , at least 16, at least 17, at least 18, at least 19, at least 20, at least 21, at least 22, at least 23, at least 24, at least 25, at least 26, at least 27, at least 28, at least 29 , at least 30, at least 31, at least 32, at least 33, at least 34, at least 35, at least 36, at least 37, at least 38 different biomarkers selected from each of Lists S1-2, S2-2 and S3-2; or

at least 4, at least 5, at least 6, at least 7, at least 8, at least 9, at least 10, at least 11, at least 12, at least 13, at least 14, at least 15, at least 16, at least 17, at least 18, at least 19, at least 20, at least 21 different biomarkers selected from each of Lists S1-3, S2-3 and S3-3.

Particularly preferred two- and three-biomarker combinations for use in the method as described above are disclosed in Table 2.

TABLE 2
Preferred combinations of two and three biomarkers

| | | | |
|---|---|---|---|
| Combinations of two | IFNG | ADGRE3 | |
| biomarkers respectively | IFNG | ARL14EP | |
| selected from Lists S1-3 and S2-3 | IFNG | CCL2 | |
| | IFNG | CCNB1IP1 | |
| | IFNG | CDKN1A | |
| | IFNG | CX3CR1 | |
| | IFNG | IFITM1 | |
| | IFNG | TGFB1 | |
| | PTGS2 | ADGRE3 | |
| | PTGS2 | ARL14EP | |
| | PTGS2 | CCL2 | |
| | PTGS2 | CCNB1IP1 | |
| | PTGS2 | CDKN1A | |
| | PTGS2 | CX3CR1 | |
| | PTGS2 | IFITM1 | |
| | PTGS2 | TGFB1 | |
| | DDX58 | ADGRE3 | |
| | DDX58 | ARL14EP | |
| | DDX58 | CCL2 | |
| | DDX58 | CCNB1IP1 | |
| | DDX58 | CDKN1A | |
| | DDX58 | CX3CR1 | |
| | DDX58 | IFITM1 | |
| | DDX58 | TGFB1 | |
| | SRC | ADGRE3 | |
| | SRC | ARL14EP | |
| | SRC | CCL2 | |
| | SRC | CCNB1IP1 | |
| | SRC | CDKN1A | |
| | SRC | CX3CR1 | |
| | SRC | IFITM1 | |
| | SRC | TGFB1 | |
| | STING | ADGRE3 | |
| | STING | ARL14EP | |
| | STING | CCL2 | |
| | STING | CCNB1IP1 | |
| | STING | CDKN1A | |
| | STING | CX3CR1 | |
| | STING | IFITM1 | |
| | STING | TGFB1 | |
| | TNFA | ADGRE3 | |
| | TNFA | ARL14EP | |
| | TNFA | CCL2 | |
| | TNFA | CCNB1IP1 | |
| | TNFA | CDKN1A | |
| | TNFA | CX3CR1 | |
| | TNFA | IFITM1 | |
| | TNFA | TGFB1 | |
| | TNFSF13B | ADGRE3 | |
| | TNFSF13B | ARL14EP | |
| | TNFSF13B | CCL2 | |
| | TNFSF13B | CCNB1IP1 | |
| | TNFSF13B | CDKN1A | |
| | TNFSF13B | CX3CR1 | |
| | TNFSF13B | IFITM1 | |
| | TNFSF13B | TGFB1 | |
| | ZBP1 | ADGRE3 | |
| | ZBP1 | ARL14EP | |
| | ZBP1 | CCL2 | |
| | ZBP1 | CCNB1IP1 | |
| | ZBP1 | CDKN1A | |
| | ZBP1 | CX3CR1 | |
| | ZBP1 | IFITM1 | |
| | ZBP1 | TGFB1 | |
| Combinations | IFNG | 121601901-HERV0116 | |
| of two | IFNG | CCR1 | |
| biomarkers | IFNG | EIF2AK4 | |
| respectively | IFNG | HLA-DPA1 | |
| selected from | IFNG | IL2 | |
| Lists S1-3 and | PTGS2 | 121601901-HERV0116 | |
| S3-3 | PTGS2 | CCR1 | |
| | PTGS2 | EIF2AK4 | |
| | PTGS2 | HLA-DPA1 | |
| | PTGS2 | IL2 | |
| | DDX58 | 121601901-HERV0116 | |
| | DDX58 | CCR1 | |
| | DDX58 | EIF2AK4 | |
| | DDX58 | HLA-DPA1 | |
| | DDX58 | IL2 | |
| | SRC | 121601901-HERV0116 | |
| | SRC | CCR1 | |
| | SRC | EIF2AK4 | |
| | SRC | HLA-DPA1 | |
| | SRC | IL2 | |
| | STING | 121601901-HERV0116 | |
| | STING | CCR1 | |
| | STING | EIF2AK4 | |
| | STING | HLA-DPA1 | |
| | STING | IL2 | |
| | TNFA | 121601901-HERV0116 | |
| | TNFA | CCR1 | |
| | TNFA | EIF2AK4 | |
| | TNFA | HLA-DPA1 | |
| | TNFA | IL2 | |
| | TNFSF13B | 121601901-HERV0116 | |
| | TNFSF13B | CCR1 | |
| | TNFSF13B | EIF2AK4 | |
| | TNFSF13B | HLA-DPA1 | |
| | TNFSF13B | IL2 | |
| | ZBP1 | 121601901-HERV0116 | |
| | ZBP1 | CCR1 | |
| | ZBP1 | EIF2AK4 | |
| | ZBP1 | HLA-DPA1 | |
| | ZBP1 | IL2 | |
| Combinations | ADGRE3 | 121601901-HERV0116 | |
| of two | ADGRE3 | CCR1 | |
| biomarkers | ADGRE3 | EIF2AK4 | |
| respectively | ADGRE3 | HLA-DPA1 | |
| selected from | ADGRE3 | IL2 | |
| Lists S2-3 and | ARL14EP | 121601901-HERV0116 | |
| S3-3 | ARL14EP | CCR1 | |
| | ARL14EP | EIF2AK4 | |
| | ARL14EP | HLA-DPA1 | |
| | ARL14EP | IL2 | |
| | CCL2 | 121601901-HERV0116 | |
| | CCL2 | CCR1 | |
| | CCL2 | EIF2AK4 | |
| | CCL2 | HLA-DPA1 | |
| | CCL2 | IL2 | |
| | CCNB1IP1 | 121601901-HERV0116 | |
| | CCNB1IP1 | CCR1 | |
| | CCNB1IP1 | EIF2AK4 | |
| | CCNB1IP1 | HLA-DPA1 | |
| | CCNB1IP1 | IL2 | |
| | CDKN1A | 121601901-HERV0116 | |
| | CDKN1A | CCR1 | |
| | CDKN1A | EIF2AK4 | |
| | CDKN1A | HLA-DPA1 | |
| | CDKN1A | IL2 | |
| | CX3CR1 | 121601901-HERV0116 | |
| | CX3CR1 | CCR1 | |
| | CX3CR1 | EIF2AK4 | |
| | CX3CR1 | HLA-DPA1 | |
| | CX3CR1 | IL2 | |
| | IFITM1 | 121601901-HERV0116 | |
| | IFITM1 | CCR1 | |
| | IFITM1 | EIF2AK4 | |
| | IFITM1 | HLA-DPA1 | |
| | IFITM1 | IL2 | |
| | TGFB1 | 121601901-HERV0116 | |
| | TGFB1 | CCR1 | |
| | TGFB1 | EIF2AK4 | |
| | TGFB1 | HLA-DPA1 | |
| | TGFB1 | IL2 | |
| Combinations | IFNG | ADGRE3 | 121601901-HERV0116 |
| of three | IFNG | ADGRE3 | CCR1 |
| biomarkers | IFNG | ADGRE3 | EIF2AK4 |
| respectively | IFNG | ADGRE3 | HLA-DPA1 |
| selected from | IFNG | ADGRE3 | IL2 |
| each of Lists | IFNG | ARL14EP | 121601901-HERV0116 |
| S1-3, S2-3 and | IFNG | ARL14EP | CCR1 |
| S3-3 | IFNG | ARL14EP | EIF2AK4 |
| | IFNG | ARL14EP | HLA-DPA1 |
| | IFNG | ARL14EP | IL2 |
| | IFNG | CCL2 | 121601901-HERV0116 |
| | IFNG | CCL2 | CCR1 |
| | IFNG | CCL2 | EIF2AK4 |
| | IFNG | CCL2 | HLA-DPA1 |
| | IFNG | CCL2 | IL2 |
| | IFNG | CCNB1IP1 | 121601901-HERV0116 |
| | IFNG | CCNB1IP1 | CCR1 |
| | IFNG | CCNB1IP1 | EIF2AK4 |
| | IFNG | CCNB1IP1 | HLA-DPA1 |
| | IFNG | CCNB1IP1 | IL2 |
| | IFNG | CDKN1A | 121601901-HERV0116 |
| | IFNG | CDKN1A | CCR1 |
| | IFNG | CDKN1A | EIF2AK4 |
| | IFNG | CDKN1A | HLA-DPA1 |
| | IFNG | CDKN1A | IL2 |
| | IFNG | CX3CR1 | 121601901-HERV0116 |
| | IFNG | CX3CR1 | CCR1 |
| | IFNG | CX3CR1 | EIF2AK4 |
| | IFNG | CX3CR1 | HLA-DPA1 |
| | IFNG | CX3CR1 | IL2 |
| | IFNG | IFITM1 | 121601901-HERV0116 |
| | IFNG | IFITM1 | CCR1 |
| | IFNG | IFITM1 | EIF2AK4 |
| | IFNG | IFITM1 | HLA-DPA1 |
| | IFNG | IFITM1 | IL2 |
| | IFNG | TGFB1 | 121601901-HERV0116 |
| | IFNG | TGFB1 | CCR1 |
| | IFNG | TGFB1 | EIF2AK4 |
| | IFNG | TGFB1 | HLA-DPA1 |
| | IFNG | TGFB1 | IL2 |
| | PTGS2 | ADGRE3 | 121601901-HERV0116 |
| | PTGS2 | ADGRE3 | CCR1 |
| | PTGS2 | ADGRE3 | EIF2AK4 |
| | PTGS2 | ADGRE3 | HLA-DPA1 |
| | PTGS2 | ADGRE3 | IL2 |
| | PTGS2 | ARL14EP | 121601901-HERV0116 |
| | PTGS2 | ARL14EP | CCR1 |
| | PTGS2 | ARL14EP | EIF2AK4 |
| | PTGS2 | ARL14EP | HLA-DPA1 |
| | PTGS2 | ARL14EP | IL2 |
| | PTGS2 | CCL2 | 121601901-HERV0116 |
| | PTGS2 | CCL2 | CCR1 |
| | PTGS2 | CCL2 | EIF2AK4 |
| | PTGS2 | CCL2 | HLA-DPA1 |
| | PTGS2 | CCL2 | IL2 |
| | PTGS2 | CCNB1IP1 | 121601901-HERV0116 |
| | PTGS2 | CCNB1IP1 | CCR1 |
| | PTGS2 | CCNB1IP1 | EIF2AK4 |
| | PTGS2 | CCNB1IP1 | HLA-DPA1 |
| | PTGS2 | CCNB1IP1 | IL2 |
| | PTGS2 | CDKN1A | 121601901-HERV0116 |
| | PTGS2 | CDKN1A | CCR1 |
| | PTGS2 | CDKN1A | EIF2AK4 |
| | PTGS2 | CDKN1A | HLA-DPA1 |
| | PTGS2 | CDKN1A | IL2 |
| | PTGS2 | CX3CR1 | 121601901-HERV0116 |
| | PTGS2 | CX3CR1 | CCR1 |
| | PTGS2 | CX3CR1 | EIF2AK4 |
| | PTGS2 | CX3CR1 | HLA-DPA1 |
| | PTGS2 | CX3CR1 | IL2 |
| | PTGS2 | IFITM1 | 121601901-HERV0116 |
| | PTGS2 | IFITM1 | CCR1 |
| | PTGS2 | IFITM1 | EIF2AK4 |
| | PTGS2 | IFITM1 | HLA-DPA1 |
| | PTGS2 | IFITM1 | IL2 |
| | PTGS2 | TGFB1 | 121601901-HERV0116 |
| | PTGS2 | TGFB1 | CCR1 |
| | PTGS2 | TGFB1 | EIF2AK4 |
| | PTGS2 | TGFB1 | HLA-DPA1 |
| | PTGS2 | TGFB1 | IL2 |
| | DDX58 | ADGRE3 | 121601901-HERV0116 |
| | DDX58 | ADGRE3 | CCR1 |
| | DDX58 | ADGRE3 | EIF2AK4 |
| | DDX58 | ADGRE3 | HLA-DPA1 |
| | DDX58 | ADGRE3 | IL2 |
| | DDX58 | ARL14EP | 121601901-HERV0116 |
| | DDX58 | ARL14EP | CCR1 |
| | DDX58 | ARL14EP | EIF2AK4 |
| | DDX58 | ARL14EP | HLA-DPA1 |
| | DDX58 | ARL14EP | IL2 |
| | DDX58 | CCL2 | 121601901-HERV0116 |
| | DDX58 | CCL2 | CCR1 |
| | DDX58 | CCL2 | EIF2AK4 |
| | DDX58 | CCL2 | HLA-DPA1 |
| | DDX58 | CCL2 | IL2 |
| | DDX58 | CCNB1IP1 | 121601901-HERV0116 |
| | DDX58 | CCNB1IP1 | CCR1 |
| | DDX58 | CCNB1IP1 | EIF2AK4 |
| | DDX58 | CCNB1IP1 | HLA-DPA1 |
| | DDX58 | CCNB1IP1 | IL2 |
| | DDX58 | CDKN1A | 121601901-HERV0116 |
| | DDX58 | CDKN1A | CCR1 |
| | DDX58 | CDKN1A | EIF2AK4 |
| | DDX58 | CDKN1A | HLA-DPA1 |
| | DDX58 | CDKN1A | IL2 |
| | DDX58 | CX3CR1 | 121601901-HERV0116 |
| | DDX58 | CX3CR1 | CCR1 |
| | DDX58 | CX3CR1 | EIF2AK4 |
| | DDX58 | CX3CR1 | HLA-DPA1 |
| | DDX58 | CX3CR1 | IL2 |
| | DDX58 | IFITM1 | 121601901-HERV0116 |
| | DDX58 | IFITM1 | CCR1 |
| | DDX58 | IFITM1 | EIF2AK4 |
| | DDX58 | IFITM1 | HLA-DPA1 |
| | DDX58 | IFITM1 | IL2 |
| | DDX58 | TGFB1 | 121601901-HERV0116 |
| | DDX58 | TGFB1 | CCR1 |
| | DDX58 | TGFB1 | EIF2AK4 |
| | DDX58 | TGFB1 | HLA-DPA1 |
| | DDX58 | TGFB1 | IL2 |
| | SRC | ADGRE3 | 121601901-HERV0116 |
| | SRC | ADGRE3 | CCR1 |
| | SRC | ADGRE3 | EIF2AK4 |
| | SRC | ADGRE3 | HLA-DPA1 |
| | SRC | ADGRE3 | IL2 |
| | SRC | ARL14EP | 121601901-HERV0116 |
| | SRC | ARL14EP | CCR1 |
| | SRC | ARL14EP | EIF2AK4 |
| | SRC | ARL14EP | HLA-DPA1 |
| | SRC | ARL14EP | IL2 |
| | SRC | CCL2 | 121601901-HERV0116 |
| | SRC | CCL2 | CCR1 |
| | SRC | CCL2 | EIF2AK4 |
| | SRC | CCL2 | HLA-DPA1 |
| | SRC | CCL2 | IL2 |
| | SRC | CCNB1IP1 | 121601901-HERV0116 |
| | SRC | CCNB1IP1 | CCR1 |
| | SRC | CCNB1IP1 | EIF2AK4 |
| | SRC | CCNB1IP1 | HLA-DPA1 |
| | SRC | CCNB1IP1 | IL2 |
| | SRC | CDKN1A | 121601901-HERV0116 |
| | SRC | CDKN1A | CCR1 |
| | SRC | CDKN1A | EIF2AK4 |
| | SRC | CDKN1A | HLA-DPA1 |
| | SRC | CDKN1A | IL2 |
| | SRC | CX3CR1 | 121601901-HERV0116 |
| | SRC | CX3CR1 | CCR1 |
| | SRC | CX3CR1 | EIF2AK4 |
| | SRC | CX3CR1 | HLA-DPA1 |
| | SRC | CX3CR1 | IL2 |
| | SRC | IFITM1 | 121601901-HERV0116 |
| | SRC | IFITM1 | CCR1 |
| | SRC | IFITM1 | EIF2AK4 |
| | SRC | IFITM1 | HLA-DPA1 |
| | SRC | IFITM1 | IL2 |
| | SRC | TGFB1 | 121601901-HERV0116 |
| | SRC | TGFB1 | CCR1 |
| | SRC | TGFB1 | EIF2AK4 |
| | SRC | TGFB1 | HLA-DPA1 |
| | SRC | TGFB1 | IL2 |
| | STING | ADGRE3 | 121601901-HERV0116 |
| | STING | ADGRE3 | CCR1 |
| | STING | ADGRE3 | EIF2AK4 |
| | STING | ADGRE3 | HLA-DPA1 |
| | STING | ADGRE3 | IL2 |
| | STING | ARL14EP | 121601901-HERV0116 |
| | STING | ARL14EP | CCR1 |
| | STING | ARL14EP | EIF2AK4 |
| | STING | ARL14EP | HLA-DPA1 |
| | STING | ARL14EP | IL2 |
| | STING | CCL2 | 121601901-HERV0116 |
| | STING | CCL2 | CCR1 |
| | STING | CCL2 | EIF2AK4 |
| | STING | CCL2 | HLA-DPA1 |
| | STING | CCL2 | IL2 |
| | STING | CCNB1IP1 | 121601901-HERV0116 |
| | STING | CCNB1IP1 | CCR1 |
| | STING | CCNB1IP1 | EIF2AK4 |
| | STING | CCNB1IP1 | HLA-DPA1 |
| | STING | CCNB1IP1 | IL2 |
| | STING | CDKN1A | 121601901-HERV0116 |
| | STING | CDKN1A | CCR1 |
| | STING | CDKN1A | EIF2AK4 |
| | STING | CDKN1A | HLA-DPA1 |
| | STING | CDKN1A | IL2 |
| | STING | CX3CR1 | 121601901-HERV0116 |
| | STING | CX3CR1 | CCR1 |
| | STING | CX3CR1 | EIF2AK4 |
| | STING | CX3CR1 | HLA-DPA1 |
| | STING | CX3CR1 | IL2 |
| | STING | IFITM1 | 121601901-HERV0116 |
| | STING | IFITM1 | CCR1 |
| | STING | IFITM1 | EIF2AK4 |
| | STING | IFITM1 | HLA-DPA1 |
| | STING | IFITM1 | IL2 |
| | STING | TGFB1 | 121601901-HERV0116 |
| | STING | TGFB1 | CCR1 |
| | STING | TGFB1 | EIF2AK4 |
| | STING | TGFB1 | HLA-DPA1 |
| | STING | TGFB1 | IL2 |
| | TNFA | ADGRE3 | 121601901-HERV0116 |
| | TNFA | ADGRE3 | CCR1 |
| | TNFA | ADGRE3 | EIF2AK4 |
| | TNFA | ADGRE3 | HLA-DPA1 |
| | TNFA | ADGRE3 | IL2 |
| | TNFA | ARL14EP | 121601901-HERV0116 |
| | TNFA | ARL14EP | CCR1 |
| | TNFA | ARL14EP | EIF2AK4 |
| | TNFA | ARL14EP | HLA-DPA1 |
| | TNFA | ARL14EP | IL2 |
| | TNFA | CCL2 | 121601901-HERV0116 |
| | TNFA | CCL2 | CCR1 |
| | TNFA | CCL2 | EIF2AK4 |
| | TNFA | CCL2 | HLA-DPA1 |
| | TNFA | CCL2 | IL2 |
| | TNFA | CCNB1IP1 | 121601901-HERV0116 |
| | TNFA | CCNB1IP1 | CCR1 |
| | TNFA | CCNB1IP1 | EIF2AK4 |
| | TNFA | CCNB1IP1 | HLA-DPA1 |
| | TNFA | CCNB1IP1 | IL2 |
| | TNFA | CDKN1A | 121601901-HERV0116 |
| | TNFA | CDKN1A | CCR1 |
| | TNFA | CDKN1A | EIF2AK4 |
| | TNFA | CDKN1A | HLA-DPA1 |
| | TNFA | CDKN1A | IL2 |
| | TNFA | CX3CR1 | 121601901-HERV0116 |
| | TNFA | CX3CR1 | CCR1 |
| | TNFA | CX3CR1 | EIF2AK4 |
| | TNFA | CX3CR1 | HLA-DPA1 |
| | TNFA | CX3CR1 | IL2 |
| | TNFA | IFITM1 | 121601901-HERV0116 |
| | TNFA | IFITM1 | CCR1 |
| | TNFA | IFITM1 | EIF2AK4 |
| | TNFA | IFITM1 | HLA-DPA1 |
| | TNFA | IFITM1 | IL2 |
| | TNFA | TGFB1 | 121601901-HERV0116 |
| | TNFA | TGFB1 | CCR1 |
| | TNFA | TGFB1 | EIF2AK4 |
| | TNFA | TGFB1 | HLA-DPA1 |
| | TNFA | TGFB1 | IL2 |
| | TNFSF13B | ADGRE3 | 121601901-HERV0116 |
| | TNFSF13B | ADGRE3 | CCR1 |
| | TNFSF13B | ADGRE3 | EIF2AK4 |
| | TNFSF13B | ADGRE3 | HLA-DPA1 |
| | TNFSF13B | ADGRE3 | IL2 |
| | TNFSF13B | ARL14EP | 121601901-HERV0116 |
| | TNFSF13B | ARL14EP | CCR1 |
| | TNFSF13B | ARL14EP | EIF2AK4 |
| | TNFSF13B | ARL14EP | HLA-DPA1 |
| | TNFSF13B | ARL14EP | IL2 |
| | TNFSF13B | CCL2 | 121601901-HERV0116 |
| | TNFSF13B | CCL2 | CCR1 |
| | TNFSF13B | CCL2 | EIF2AK4 |
| | TNFSF13B | CCL2 | HLA-DPA1 |
| | TNFSF13B | CCL2 | IL2 |
| | TNFSF13B | CCNB1IP1 | 121601901-HERV0116 |
| | TNFSF13B | CCNB1IP1 | CCR1 |
| | TNFSF13B | CCNB1IP1 | EIF2AK4 |
| | TNFSF13B | CCNB1IP1 | HLA-DPA1 |
| | TNFSF13B | CCNB1IP1 | IL2 |
| | TNFSF13B | CDKN1A | 121601901-HERV0116 |
| | TNFSF13B | CDKN1A | CCR1 |
| | TNFSF13B | CDKN1A | EIF2AK4 |
| | TNFSF13B | CDKN1A | HLA-DPA1 |
| | TNFSF13B | CDKN1A | IL2 |
| | TNFSF13B | CX3CR1 | 121601901-HERV0116 |
| | TNFSF13B | CX3CR1 | CCR1 |
| | TNFSF13B | CX3CR1 | EIF2AK4 |
| | TNFSF13B | CX3CR1 | HLA-DPA1 |
| | TNFSF13B | CX3CR1 | IL2 |
| | TNFSF13B | IFITM1 | 121601901-HERV0116 |
| | TNFSF13B | IFITM1 | CCR1 |
| | TNFSF13B | IFITM1 | EIF2AK4 |
| | TNFSF13B | IFITM1 | HLA-DPA1 |
| | TNFSF13B | IFITM1 | IL2 |
| | TNFSF13B | TGFB1 | 121601901-HERV0116 |
| | TNFSF13B | TGFB1 | CCR1 |
| | TNFSF13B | TGFB1 | EIF2AK4 |
| | TNFSF13B | TGFB1 | HLA-DPA1 |
| | TNFSF13B | TGFB1 | IL2 |
| | ZBP1 | ADGRE3 | 121601901-HERV0116 |
| | ZBP1 | ADGRE3 | CCR1 |
| | ZBP1 | ADGRE3 | EIF2AK4 |
| | ZBP1 | ADGRE3 | HLA-DPA1 |
| | ZBP1 | ADGRE3 | IL2 |
| | ZBP1 | ARL14EP | 121601901-HERV0116 |
| | ZBP1 | ARL14EP | CCR1 |
| | ZBP1 | ARL14EP | EIF2AK4 |
| | ZBP1 | ARL14EP | HLA-DPA1 |
| | ZBP1 | ARL14EP | IL2 |
| | ZBP1 | CCL2 | 121601901-HERV0116 |
| | ZBP1 | CCL2 | CCR1 |
| | ZBP1 | CCL2 | EIF2AK4 |
| | ZBP1 | CCL2 | HLA-DPA1 |
| | ZBP1 | CCL2 | IL2 |
| | ZBP1 | CCNB1IP1 | 121601901-HERV0116 |
| | ZBP1 | CCNB1IP1 | CCR1 |
| | ZBP1 | CCNB1IP1 | EIF2AK4 |
| | ZBP1 | CCNB1IP1 | HLA-DPA1 |
| | ZBP1 | CCNB1IP1 | IL2 |
| | ZBP1 | CDKN1A | 121601901-HERV0116 |
| | ZBP1 | CDKN1A | CCR1 |
| | ZBP1 | CDKN1A | EIF2AK4 |
| | ZBP1 | CDKN1A | HLA-DPA1 |
| | ZBP1 | CDKN1A | IL2 |
| | ZBP1 | CX3CR1 | 121601901-HERV0116 |
| | ZBP1 | CX3CR1 | CCR1 |
| | ZBP1 | CX3CR1 | EIF2AK4 |
| | ZBP1 | CX3CR1 | HLA-DPA1 |
| | ZBP1 | CX3CR1 | IL2 |
| | ZBP1 | IFITM1 | 121601901-HERV0116 |
| | ZBP1 | IFITM1 | CCR1 |
| | ZBP1 | IFITM1 | EIF2AK4 |
| | ZBP1 | IFITM1 | HLA-DPA1 |
| | ZBP1 | IFITM1 | IL2 |
| | ZBP1 | TGFB1 | 121601901-HERV0116 |
| | ZBP1 | TGFB1 | CCR1 |
| | ZBP1 | TGFB1 | EIF2AK4 |
| | ZBP1 | TGFB1 | HLA-DPA1 |
| | ZBP1 | TGFB1 | IL2 |

Preferably, the method as described above, in all its embodiments, is applied to a blood sample from a patient, preferably a patient in the hospital, more preferably a patient in the emergency department, a resuscitation unit, intensive care unit or continuous care unit, even more preferably a patient suffering from trauma (preferably, severe trauma), burns (preferably, severe burn), had surgery (in particular major surgery) or in a septic state, and very particularly preferably a patient in septic shock. By a sepsis patient is meant a patient with at least one life-threatening organ failure caused by an inappropriate host response to an infection. By septic shock is meant a subtype of sepsis, in which hypotension persists, despite adequate vascular filling.

Preferably, the method as described previously, in all its embodiments, is applied to a blood sample containing leukocytes. The blood sample can for example be a sample of peripheral blood mononuclear cells (or PBMC), which consists of lymphocytes (B, T and NK cells), dendritic cells and monocytes, and which is generally obtained by the Ficoll method, well known to one skilled in the art. However, in a particularly advantageous manner, it will be preferred to use directly a sample of whole blood (that is to say containing all the leukocytes, erythrocytes, platelets and plasma), as collected by the venous route (for example in using tubes containing an anticoagulant), in order to minimize manipulations of the sample and to preserve the physiological cellular interactions between the different cell populations involved in the immune response, and to better reflect the complexity of the innate and adaptive immune responses in the individual. In particular, while PBMCs only contain mononuclear cells, whole blood also contains granulocytes (or polymorphonuclear cells). It is also particularly advantageous to use systems that allow standardization of procedures; in particular, it is possible to use semi-closed culture systems (e.g. tubes) pre-filled with the culture medium and the stimulus of interest, which are standardized, e.g. which contain a well-defined stimulus (i.e. without inter-batches at the level of the production of the stimulus, as to its nature/composition) and/or loaded in «batch», so as to control the quantity of stimulus in the tube and to have tube-to-tube reproducibility. Preferably, these tubes can also allow the collection of the blood sample (which allows the cells to be stimulated at the time of collection), and more preferably, they allow the collection of a precise volume of blood. An example of standardized systems is TruCulture® tubes.

The blood sample may have been taken at the doctor request, for example to find out if an individual will respond to a vaccine injection. The sample may also have been taken on admission or during the evolution of the patient; in particular, for patients suffering from sepsis or patients suffering from trauma, the sample may in particular have been taken during the first week (e.g. from D3 to D7, and in particular at D3/4) after the aggression (i.e. the sepsis or trauma) or after septic shock (in particular when the patient needs vasopressors and his lactate exceeds 2 mmol/L).

In the method as described above, in all its embodiments, the step of incubating the blood sample of the individual with the stimulus can be carried out at different temperatures (preferably at 37° C.) and at different incubation times (preferably between 1 hour and 48 hours of incubation; for example, with an incubation of 1 hour or less, 2 hours or less, 4 hours or less, 12 hours or less, 24 hours or less, or 48 hours or less). Short incubation times are particularly advantageous for the implementation of the test in the clinic.

The stimulus used in the method as described previously, in all its embodiments, can be of different natures.

According to an embodiment, the stimulus may comprise one (or more) molecule(s) of the immunogenic type(s). In this embodiment, the method is particularly useful for determining a diagnosis (in particular concerning the immune status of the individual), a prognosis (in particular concerning the evolution of the immune status of the individual), and/or adapting the therapeutic care of said individual.

The immunogenic-type stimulus may, for example, comprise one or more molecules capable of binding:

at least one type of antigen-presenting cell (APC), said APC possibly being in particular a type of cell of innate immunity (e.g. a monocyte, a macrophage, or a dendritic cell) or a type of cell of the adaptive immunity (e.g. a B lymphocyte), on the one hand, and

at least one type of adaptive immunity cell (such as a T lymphocyte), on the other hand.

Preferably, this stimulus comprises a molecule of the superantigen type or a molecule analogous to a superantigen. Superantigens are toxins of a protein nature, capable of stimulating a large number of T lymphocytes, through their simultaneous binding to the β chain of the variable domain (V_{β}) of a T cell receptor via the hypervariable region CDR4, and to a molecule of MHC II (class II major histocompatibility complex), present on the surface of an antigen-presenting cell (APC). The forced interaction that is established between the antigen-presenting cell carrying the MHC and the T lymphocytes whose T cell receptor carries the V_{β}segment, causes a polyclonal activation of these T lymphocytes, independently of their specificity for the presented peptide antigen. When a stimulus comprising a molecule of the superantigen type is used, the blood sample used in the method according to the invention contains T lymphocytes and antigen-presenting cells. Among the superantigens of more particular interest, mention may in particular be made of the superantigens produced by staphylococcal species and the superantigens produced by streptococcal species. Preferably, the stimulus comprises at least one molecule selected from SEB (Staphylococcal Enterotoxin B) and SEA (Staphylococcal Enterotoxin A). Among the molecules analogous to a superantigen, mention may be made, for example, of bispecific antibodies, capable of binding on the one hand to a T lymphocyte, and on the other hand to an antigen-presenting cell (such as, for example, antibodies capable of binding on the one hand to V_{β} on T lymphocytes, and on the other hand to a molecule of MHC II or to a TLR-type receptor, on antigen-presenting cells).

It can also be a stimulus which directly activates the T lymphocytes, which is preferably selected from antibodies recognizing and activating a receptor on the surface of the T lymphocyte so as to trigger an activation signal at the level of the T lymphocyte, more preferably these antibodies being associated physically and/or chemically with each other, more preferably still by coupling on polymers, by coupling on beads or by coupling between them. They may for example be anti-CD3 antibodies (such as Muromonab-CD3, marketed under the name Orthoclone OKT3), preferably associated with anti-CD28, anti-CD2 and/or anti-CD137/TNFRSF9 antibodies.

It may also be a stimulus of the imidazoquinolin type, structural analogues of a nucleoside, including a ring in their structure, of low molecular weight. This type of stimulus produces in vivo antiviral and antitumor effects. An example of an imidazoquinoline-type stimulus may be mentioned, Resiquimod (R848), which binds to human TLR7 and TLR8 on dendritic cells, or more generally on antigen-presenting cells, or APC (NF response-KB dependent). Direct effects on T lymphocytes have also been described (Smits et al (2008), Oncologist 13(8): 859-875).

According to another embodiment, the stimulus may comprise, preferably consist essentially of, more preferably consist of, a molecule for therapeutic purposes (in particular, a drug or a drug candidate), and more preferably a molecule having an immunomodulatory effect (in particular, a molecule having an immunostimulant or anti-inflammatory effect). IL-7 or interferon y may be mentioned by way of example. In this embodiment, the method is particularly useful for predicting and/or monitoring the efficacy of response to said molecule for therapeutic purposes.

Measuring the expression (or level of expression) of a biomarker consists in quantifying at least one expression product of this biomarker. The expression product of a biomarker within the meaning of the invention is any biological molecule resulting from the expression of this biomarker. More particularly, the expression product of a biomarker can be an RNA transcript. By «transcript», is meant the RNAs, and in particular the messenger RNAs (mRNAs), resulting from the transcription of the biomarker. More specifically, transcripts are RNAs produced by the transcription of a gene followed by post-transcriptional modifications of pre-RNA forms.

Thus, preferably, in the method as described previously, in all its embodiments, the expression of the biomarkers is measured at the RNA or mRNA transcript level. In the context of the present invention, the measurement of the level of expression of one or more RNA transcripts of the same biomarker can be carried out. The determination of the quantity of several transcripts can be implemented sequentially or simultaneously, according to methods well known to one skilled in the art. The detection of an mRNA transcript can be carried out by a direct method, by any method known to one skilled in the art making it possible to determine the presence of said transcript in the sample, or by indirect detection of the transcript after transformation of the latter into DNA, or after amplification of said transcript or after amplification of the DNA obtained after transformation of said transcript into DNA. Many methods exist for the detection of nucleic acids (see for example Kricka et al., Clinical Chemistry, 1999, n° 45(4), p.453-458; Relier GH et al., DNA Probes, 2nd Ed., Stockton Press, 1993, sections 5 and 6, p.173-249). The expression of the biomarkers can in particular be measured by Reverse Transcription-Polymerase Chain Reaction or RT-PCR, preferably by quantitative RT-PCR or RT-qPCR (for example using FilmArray® technology), by sequencing (preferably by sequencing high throughput) or by hybridization techniques (for example with hybridization microchips or by techniques of the NanoString® nCounter® type). Techniques allowing multiplexing (such as FilmArray® or NanoString® nCounter®) are preferred.

In the context of the present invention, the measurement of the level of expression makes it possible to determine the quantity of one or more transcripts present in the tested sample or to give a derived value therefrom. A value derived from the quantity can for example be the absolute concentration, calculated using a calibration curve obtained from successive dilutions of a solution of amplicons of known concentration. It can also correspond to the value of the standardized and calibrated quantity, such as the CNRQ (Calibrated Normalized Relative Quantity, (Hellemans et al (2007), Genome biology 8(2):R19)), which integrates the values of a sample reference, a calibrator and one or more housekeeping genes (also called reference genes). As examples of a reference gene, mention may be made of the PPIB, PPIA, GLYR1, RANBP3, HPRT1, 18S, GAPDH, RPLP0 and ACTB genes.

Preferably, in the method as previously described, in all its embodiments, the expression of the biomarkers is normalized with respect to the expression of one or more of the following reference genes: HPRT1, DECR1 and TBP; in particular, the geometric mean of the 3 genes HPRT1, DECR1 and TBP can be used for normalization.

Preferably, the method as previously described, in all its embodiments, can also comprise a step of measuring the expression, from a control blood sample without stimulation (that is to say the sample blood incubated under the same conditions as the stimulated blood sample, but in the absence of stimulus), of the same biomarkers as those measured from the stimulated blood sample. Preferably again, the method comprises a step of calculating the ratios of the expression (preferably, the normalized expression) of each biomarker in the stimulated blood sample, relative to the expression (preferably, the normalized expression), of the same biomarker in the control blood sample. Even more preferably, the method comprises a step of transforming the ratios obtained by a base logarithmic transformation 10, and possibly steps of transforming into reduced centered variables.

The invention also relates to a kit comprising means for amplifying and/or detecting (preferably primers and/or probes) at least two different biomarkers, selected respectively from at least two different lists from:

Lists S1, List S2 and List S3;

Lists S1-1, List S2-1 and List S3-1;

Lists S1-2, List S2-2 and List S3-2: or

Lists S1-3, List S2-3 and List S3-3;

preferably comprising means of amplification and/or detection (preferably primers and/or probes) of at least three different biomarkers, selected respectively from each of the three lists:

Lists S1, List S2 and List S3;

Lists S1-1, List S2-1 and List S3-1;

Lists S1-2, List S2-2 and List S3-2: or

Lists S1-3, List S2-3 and List S3-3;

more preferably comprising means of amplification and/or detection (preferably primers and/or probes):

at least 4, at least 5, at least 6, at least 7, at least 8, at least 9, at least 10, at least 11, at least 12, at least 13, at least 14, at least 15 , at least 16, at least 17, at least 18, at least 19, at least 20, at least 21, at least 22, at least 23, at least 24, at least 25, at least 26, at least 27, at least at least 28, at least 29, at least 30, at least 31, at least 32, at least 33, at least 34, at least 35, at least 36, at least 37, at least 38, at least 39, at least 40 , at least 41, at least 42, at least 43, at least 44, at least 45, at least 46 different biomarkers selected from each of Lists S1, S2 and S3;

at least 4, at least 5, at least 6, at least 7, at least 8, at least 9, at least 10, at least 11, at least 12, at least 13, at least 14, at least 15 , at least 16, at least 17, at least 18, at least 19, at least 20, at least 21, at least 22, at least 23, at least 24,

at least 25, at least 26, at least 27, at least at least 28, at least 29, at least 30, at least 31, at least 32, at least 33, at least 34, at least 35, at least 36, at least 37, at least 38, at least 39, at least 40, at least 41, at least 42, at least 43, at least 44, at least 45 different biomarkers selected from each of Lists S1-1, S2-1 and S3-1;

at least 4, at least 5, at least 6, at least 7, at least 8, at least 9, at least 10, at least 11, at least 12, at least 13, at least 14, at least 15 , at least 16, at least 17, at least 18, at least 19, at least 20, at least 21, at least 22, at least 23, at least 24, at least 25, at least 26, at least 27, at least at least 28, at least 29, at least 30, at least 31, at least 32, at least 33, at least 34, at least 35, at least 36, at least 37, at least 38 different biomarkers selected from each of Lists S1-2, S2-2 and S3-2; or

at least 4, at least 5, at least 6, at least 7, at least 8, at least 9, at least 10, at least 11, at least 12, at least 13, at least 14, at least 15, at least 16, at least 17, at least 18, at least 19, at least 20, at least 21 different biomarkers selected from each of Lists S1-3, S2-3 and S3-3;

said kit being characterized in that all of the amplification and/or detection means of said kit allow the detection and/or amplification of at most 100 (preferably at most 90, preferably at most 80, preferably at most 70, preferably at most 60, preferably at most 50, preferably at most 40, preferably at most 30, preferably at most 20, preferably at most 10, preferably at most 5) biomarkers, in total.

Thus, said kit can for example also comprise means for amplifying and/or detecting one or more housekeeping genes. The kit can also comprise positive control means making it possible to qualify the quality of the RNA extraction, the quality of any amplification and/or hybridization method.

The term «primer» or «amplification primer» means a nucleotide fragment which may consist of 5 to 100 nucleotides, preferably of 15 to 30 nucleotides, and possessing a specificity of hybridization with a target nucleotide sequence, under conditions determined for the initiation of an enzymatic polymerization, for example in an enzymatic amplification reaction of the target nucleotide sequence. Generally, «pairs of primers» are used, consisting of two primers.

When it is desired to carry out the amplification of several different biomarkers (e.g. genes), several different pairs of primers are preferably used, each preferably having the ability to be specifically hybridized with a different biomarker.

The term «probe» or «hybridization probe» means a nucleotide fragment typically consisting of 5 to 100 nucleotides, preferably of 15 to 90 nucleotides, even more preferably of 15 to 35 nucleotides, possessing a hybridization specificity under determined conditions to form a hybridization complex with a target nucleotide sequence. The probe also includes a reporter (such as a fluorophore, an enzyme or any other detection system), which will allow the detection of the target nucleotide sequence. In the present invention, the target nucleotide sequence can be a nucleotide sequence comprised in a messenger RNA (mRNA) or a nucleotide sequence comprised in a complementary DNA (cDNA) obtained by reverse transcription of said mRNA. When it is desired to target several different biomarkers (e.g. genes), several different probes are preferably used, each preferably having the ability to be hybridized specifically with a different biomarker.

The term «hybridization» means the process during which, under appropriate conditions, two nucleotide fragments, such as for example a hybridization probe and a target nucleotide fragment, having sufficiently complementary sequences, are capable of forming a double strand with stable and specific hydrogen bonds. A nucleotide fragment «capable of being hybridized» with a polynucleotide is a fragment capable of being hybridized with said polynucleotide under hybridization conditions, which can be determined in each case in a known manner. The hybridization conditions are determined by the stringency, that is to say the rigor of the operating conditions. The hybridization is all the more specific as it is carried out at higher stringency. The stringency is defined in particular according to the base composition of a probe/target duplex, as well as by the degree of mismatch between two nucleic acids. The stringency can also be a function of the reaction parameters, such as the concentration and the type of ionic species present in the hybridization solution, the nature and the concentration of denaturing agents and/or the hybridization temperature. The stringency of the conditions under which a hybridization reaction must be carried out will mainly depend on the used hybridization probes. All of these data are well known and the appropriate conditions zo can be determined by one skilled in the art. In general, depending on the length of the used hybridization probes, the temperature for the hybridization reaction is comprised between about 20 and 70° C., in particular between 35 and 65° C. in a saline solution at a concentration of about 0.5 to 1 M. A step of detecting the hybridization reaction is then carried out.

The term «enzymatic amplification reaction» means a process generating multiple copies of a target nucleotide fragment, by the action of at least one enzyme. Such amplification reactions are well known to one skilled in the art and the following techniques may be mentioned in particular: PCR (Polymerase Chain Reaction), LCR (Ligase Chain Reaction), RCR (Repair Chain Reaction), 3SR (Self Sustained Sequence Replication) with patent application WO-A-90/06995, NASBA (Nucleic Acid Sequence-Based Amplification), TMA (Transcription Mediated Amplification) with patent US-A-5,399,491, and LAMP (Loop mediated isothermal amplification) with patent US6410278. When the enzymatic amplification reaction is a PCR, we will speak more particularly of RT-PCR (RT for «reverse transcription»), when the amplification step is preceded by a messenger RNA reverse transcription step (mRNA) to complementary DNA (cDNA), and qPCR or RT-qPCR when the PCR is quantitative.

The invention also relates to the use of:

amplification and/or detection means (preferably primers and/or probes) as previously described in the kit according to the invention, in all its embodiments; preferably, means for amplifying and/or detecting (preferably primers and/or probes) at least two different biomarkers, selected respectively from at least two different lists among lists S1 to S3 (or S1-1 to S3-1, or S1-2 to S3-2, or S1-3 to S3-3), more preferably means for amplifying and/or detecting at least three different biomarkers, selected respectively from each of the three lists S1 to S3 (or S1-1 to S3-1, or S1-2 to S3-2, or S1-3 to S3-3)), or

a kit comprising such amplification and/or detection means, preferably all of the amplification and/or detection means of said kit allow the detection and/or amplification of at most 100 (preferably at most 90, preferably at most 80, preferably at most 70, preferably at most 60, preferably at most 50, preferably at most 40, preferably at most 30, preferably at most 20, preferably at most 10, preferably at most 5) biomarkers, in total, and optionally said kit comprises means for amplifying and/or detecting one or more housekeeping genes and/or positive control means making it possible to qualify the quality of RNA extraction, the quality of any amplification and/or hybridization method,

to determine an individual ability to respond to a stimulus, preferably the ability of an individual immune system to respond to a stimulus.

### FIGURES

FIG. 1: The biomarkers contributing the most to the variance for the response of healthy individuals and patients in septic shock, following stimulation with SEB. (A) Principal component analysis (PCA) of the response (stimulated sample/control sample) of 10 healthy individuals (circles) and 30 patients in septic shock (triangles), following stimulation with SEB. Each individual («donor», D) is labeled by its number. The percentage of variance explained by each Principal Component (PC) axis is indicated, as well as the total variance. The position of the vector for each individual was plotted. The most important variables are represented graphically in (B) (representing 20% of the total weight of the variables for PC1 and PC2).

FIG. 2: Multivariate clustering analysis, following stimulation with SEB. 10 healthy individuals and 30 patients with septic shock were treated as a whole, in order to discriminate gene expression profiles. The response to stimulation with SEB revealed 3 groupings or clusters (S1; n=16, S2; n=11 and S3; n=12), by using the PAM method with correlation distance (score index=31). The dendrogram is based on the distance between the individuals of the medoid of each cluster found by the PAM method. A higher intensity of the gray level (approaching black) on the thermal map (or heatmap) indicates a higher value of the expression ratio or fold change (stimulated sample/control sample) of the biomarkers and a lower intensity of the gray level (approaching white) indicates a lower value of the expression ratio or fold change (stimulated sample/control sample) of the biomarkers. The value 10,000 Ab/c was used as the threshold for high and low mHLA-DR levels. HLA-DR: human leukocyte antigen DR.

FIG. 3: Distribution of protein TNFα secretion post-stimulation with LPS and mHLA-DR by defined clusters following stimulation with SEB. At day 3-4 after the onset of septic shock, (A) secretion of protein TNFα was measured ex vivo 24 hours post-stimulation with LPS in healthy individuals (circles) and patients in septic shock (squares), and (B) mHLA-DR was measured by flow cytometry, only in patients with septic shock (squares). Mortality (non-surviving individuals) is represented by triangles and nosocomial infections by empty squares. Defined clusters post-stimulation with SEB were obtained using the PAM method with correlation distance. **p <0.001; ***p <0.0001. SEB: staphylococcal enterotoxin B, LPS: lipopolysaccharide, mHLA-DR: monocyte human leukocyte antigen DR.

The present invention is illustrated without limitation by the following examples.

### EXAMPLES

### Materials and Methods

Population of Tested Individuals

The clinical study was approved by the regional ethics committee (Comite de Protection des Hommes Sud-Est II, number 11236), and registered with the French Ministry of Research (Ministere de l′Enseignement supérieur, de la Recherche et de I'Innovation; DC-2008-509) and the National Data Protection Commission (Commission Nationale de l′Informatique et des Libertés). This study was conducted on patients with septic shock admitted to the intensive care unit of Edouard Herriot Hospital (Hospices Civils de Lyon, Lyon, France) and is part of a larger study looking at immune dysfunctions related in the intensive care unit (NCT02803346).

Patients with septic shock were included prospectively. Septic shock has been defined according to the Sepsis-3 consensus of the Society for Critical Care Medicine and the European Society for Critical Care Medicine (Singer et al (2016), JAMA 315:801-10): patients requiring the administration of a vasopressor and having a measurement of the serum lactate concentration greater than 2 mmol/L in the absence of hypovolemia in a patient having an infection, or suspected of having an infection (i.e. criteria which define the onset of septic shock in a patient with sepsis). The exclusion criteria were an age below 18 years and the presence of aplasia or a known immunosuppressive disease. At admission, the collected data included demographic characteristics (age, gender) and site of primary infection; the initial severity was assessed by the simplified severity index (IGS II; range of values: 0-163) on admission. Information regarding death during ICU stay was collected, and severity 24 hours after admission was assessed by Sequential Organ Failure Assessment (SOFA) score (range of values: 0-24). Laboratory data during follow-up were also collected, including monocyte HLA-DR (mHLA-DR) values, as well as measurement of TNFα protein secretion after LPS stimulation.

At the same time, blood samples from healthy individuals (or healthy volunteers) were obtained from the national blood service (French Blood Establishment) and immediately used.

Immune Functional Tests

Incubation in TruCulture Tubes

Heparinized whole blood (1 mL) from patients in septic shock, collected on days 3-4 after onset of septic shock, or from healthy individuals, was dispensed into TruCulture tubes (Myriad Rbm, Austin, Tex., United States) prewarmed, containing medium alone («control sample») or medium with SEB (400 ng/mL). These tubes were then inserted into a dry block incubator and maintained at 37° C. for 24 hours. After incubation, the cell pellet was resuspended in 2 ml of TRI Reagent® LS (Sigma-Aldrich, Deisenhofen, Germany), vortexed for 2 minutes and allowed to stand for 10 minutes at room temperature, before storage at room temperature −80° C.

Measurement of the Expression of Biomarkers

For TruCulture cell pellet manipulation and RNA processing and detection, the protocol was followed according to Urrutia et al (2016), Cell Reports 16, 2777-2791. The cell pellets originating from the stimulations by TruCulture and preserved in the TRI Reagent° LS (Sigma-Aldrich) were thawed under shaking. Prior to processing, thawed samples were centrifuged (at 3000g for 5 minutes at 4° C.) to sediment cellular debris generated during Trizol lysis. For extraction, a modified protocol of the NucleoSpin 96 RNA tissue kit (Macherey-Nagel Gmbh&Co. KG, Düren, Germany) was followed using a vacuum system. Briefly, 600 μl of clear lysate obtained by Trizol lysis was transferred to a tube preloaded with 900 μl of 100% ethanol.

The mixture was transferred to a silica column, then washed with buffers MW1 and MW2, and RNA was eluted using 30 μL of RNase-free water. Nanostring technology was used for mRNA detection of a panel of 46 biomarkers (Table 3)—this is a hybridization-based multiplex assay characterized by the absence of an amplification; 300 ng of RNA were hybridized to the probes at 67° C. for 18 hours using a thermocycler (Biometra, Tprofesssional TRIO, Analytik Jena AG, Jena, Germany).

After removal of excess probes, samples were loaded into nCounter Prep Station (NanoString Technologies, Seattle, Wash., USA) for purification and immobilization on the inner surface of a sample cartridge for 2-3 time. The sample cartridge was then transferred and imaged on the nCounter Digital Analyzer (NanoString Technologies) where the color codes were counted and tabulated for the 46 biomarkers

TABLE 3
Target biomarkers used for the Nanostring ® nCounter ®,
and their accession number (or chromosomal location)

| | Accession number or |
|---|---|
| Target biomarkers | chromosomal location |
| ADGRE3 | NM_032571.2 |
| ARL14EP | NM_152316.1 |
| BST2 | NM_004335.2 |
| C3 | NM_000064.2 |
| CCL2 | NM_002982.3 |
| CCL20 | NM_004591.1 |
| CCL4 | NM_002984.2 |
| CCL8 | NM_005623.2 |
| CCNB1IP1 | NM_182849.2 |
| CCR1 | NM_001295.2 |
| CD209 | NM_021155.2 |
| CD3D | NM_000732.4 |
| CD44 | NM_001001392.1 |
| CD74 | NM_001025159.1 |
| CD83 | NM_004233.3 |
| CDKN1A | NM_000389.2 |
| CLEC7A | NM_197954.2 |
| CX3CR1 | NM_001337.3 |
| CXCL10 | NM_001565.1 |
| CXCL2 | NM_002089.3 |
| CXCL9 | NM_002416.1 |
| DDX58 | NM_014314.3 |
| DYRK2 | NM_003583.3 |
| EIF2AK4 | NM_001013703.2 |
| FAM89A | NM_198552.2 |
| HLA-DMB | NM_002118.3 |
| HLA-DPA1 | NM_033554.2 |
| HLA-DPB1 | NM_002121.4 |
| HLA-DRA | NM_019111.3 |
| IFITM1 | NM_003641.3 |
| IFNG | NM_000619.2 |
| IL1A | NM_000575.3 |
| IL2 | NM_000586.2 |
| IL7R | NM_002185.2 |
| IRAK2 | NM_001570.3 |
| PTGS2 | NM_000963.1 |
| RARRES3 | NM_004585.3 |
| SLAMF7 | NM_021181.3 |
| SRC | NM_005417.3 |
| STAT2 | NM_005419.2 |
| STING | NM_198282.1 |
| TGFB1 | NM_000660.3 |
| TNFA | NM_000594.2 |
| TNFSF13B | NM_006573.4 |
| ZBP1 | NM_001160419.2 |
| 121601901-HERV0116 | chr12:112972627-112975754 |

Generation of Normalized Data

Each sample was analyzed in a separate multiplexed reaction each comprising 8 negative probes and 6 serial concentrations of positive control probes. Negative control analysis was performed to determine background for each sample. Data were imported into nSolver analysis software (version 4.0, NanoString Technologies) for quality control and data normalization.

A first standardization step using inner positive controls allows correcting the potential source of variation associated with the technical platform. To do this, we calculated for all samples the level of the average background noise as being the median +3 standard deviations of all six negative probes. Each sample below the background noise level was set to this value.

Then, the geometric mean of the positive probes is calculated for each sample. A scale factor for a sample was a ratio of the geometric mean of the sample and the mean of all the geometric means. For each sample, all gene values are divided by the corresponding scale factor.

Finally, to normalize the differences in the amount of introduced RNA, the same method as in normalization by positive controls is used, except that geometric means were calculated for three housekeeping genes (HPRT1 (NM_000194.1), DECR1 (NM_001359.1) and TBP (NM_001172085.1)).

These genes were selected using the NormFinder method, an established approach for the identification of stable intra- and inter-group housekeeping genes, from the 6 candidate genes included in the custom gene panel. The results are expressed as an expression ratio (or «fold change»). A TruCulture tube containing SEB failed quality control and was not included in the analysis.

Measurement of mHLA-DR Expression by Flow Cytometry

The expression of HLA-DR on the surface of circulating monocytes (mHLA-DR) of patients was evaluated at days 3-4 after the onset of septic shock, on peripheral whole blood collected in EDTA tubes, by flow cytometry (NAVIOS; Beckman-Coulter, Brea, Calif., USA). The results are expressed as the number of antibodies bound per cell (Ab/C).

Protein Detection

TNFα protein in the supernatant of TruCulture tubes was quantified, for septic shock patients and healthy individuals, using the ELLA nanofluidic system (Biotechne, Minneapolis, Mich., USA), in accordance with the manufacturer instructions. The results are expressed in pg/ml.

Statistical Analysis

Results are expressed as median and interquartile ranges [IQR] for continuous variables. Parametric data were analyzed by ANOVA and non-parametric data were analyzed by Kruskal-Wallis test. Statistical analyzes were conducted using GraphPad Prism® software (version 5; GraphPad software, La Jolla, Calif., USA) and R (version 3.5.1). An adjusted p-value <0.05 was considered statistically significant. Principal component analysis (PCA) was performed using Genomics Suite 7 (Partek, St. Louis, Mo., USA).

Creation of Clusters

The data were transformed by a base logarithmic transformation 10, centered and reduced. Two distance matrices and a correlation matrix were built on the data and 10 clustering methods were launched («hierarchical», «kmeans», «diana», «fanny», «som», «model», «sota», «pam», «clara» and «agnes»). For each method, k=3 to k=18 clusters were tested. The best clustering methods were selected using 7 indices combining internal measures (connectivity, silhouette width and Dunn's indice) and stability (average proportion of nonoverlapping (APN), average distance (AD), average distance between means (ADM) and figure of merit (FOM)). The most stable method for SEB was selected: it is the PAM method using the correlation matrix (score index=31).

Results

Diversity of Response to Stimulation with SEB

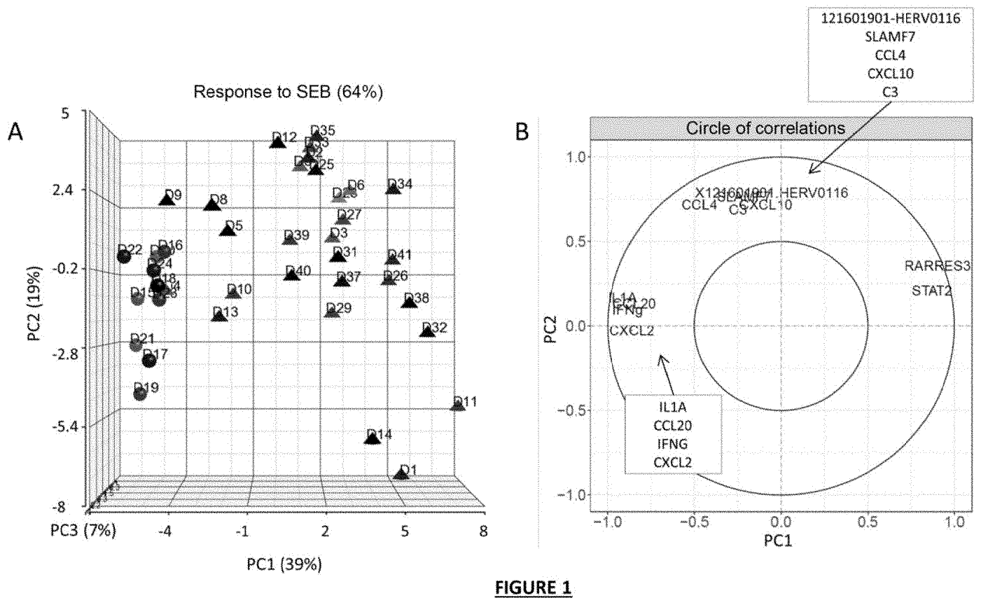

In order to identify the biomarkers contributing mainly to the quantitative variation of the response to stimulation by SEB (FIG. 1A) for healthy individuals and for patients in septic shock, these were represented graphically and the weight of the biomarkers explaining the variance was obtained (Table 4). Among the largest contributors to SEB response variance (FIG. 1B) for the first component PC1 (39%), RARRES3 and STAT2 were found to be most strongly expressed by individuals on the right side of the component, while IL1A, CXCL2 and IFNG were more strongly expressed by individuals on the opposite side. Regarding the second component PC2 (19%), the variance was induced «in the lead» by an element of human endogenous retrovirus or HERV (121601901-HERV0116), but also by SLAMF7, CCL4, C3 and CXCL10.

TABLE 4
Weights of the biomarkers responsible
for the greatest variance of the first component
(PC1) and the second component (PC2) for stimulation
by SEB in the two populations. For each
component, the biomarkers were ranked, from
the highest weight (in absolute value) to
the lowest weight (in absolute value).

| PC1 | Weight | PC2 | Weight |
|---|---|---|---|
| IL1A | −0.2167 | 121601901- | 0.2687 |
| | | HERV0116 | |
| RARRES3 | 0.2137 | SLAMF7 | 0.2608 |
| IFNG | −0.2097 | CCL4 | 0.2454 |
| STAT2 | 0.2065 | CXCL10 | 0.2445 |
| CXCL2 | −0.2039 | C3 | 0.2345 |
| CCL20 | −0.2006 | CD74 | 0.2344 |
| CD209 | −0.1965 | HLA-DRA | 0.2285 |
| PTGS2 | −0.1964 | CXCL9 | 0.2239 |
| ZBP1 | 0.1940 | HLA-DPA1 | 0.2208 |
| CD83 | −0.1866 | ADGRE3 | −0.2049 |
| CDKN1A | −0.1833 | CD44 | 0.1968 |
| DDX58 | 0.1811 | HLA-DPB1 | 0.1942 |
| HLA-DMB | 0.1798 | HLA-DMB | 0.1858 |
| CX3CR1 | 0.1782 | TNFSF13B | 0.1805 |
| BST2 | 0.1774 | IRAK2 | 0.1726 |
| IL2 | −0.1721 | SRC | 0.1637 |
| TNFA | −0.1717 | TNFA | 0.1574 |
| IRAK2 | −0.1706 | BST2 | 0.1489 |
| HLA-DPB1 | 0.1654 | STING | 0.1447 |
| CCL2 | −0.1623 | FAM89A | 0.1350 |
| HLA-DRA | 0.1513 | RARRES3 | 0.1221 |
| HLA-DPA1 | 0.1491 | CD83 | 0.1181 |
| CD74 | 0.1462 | DDX58 | 0.1069 |
| IFITM1 | 0.1449 | CCL8 | 0.1066 |
| DYRK2 | 0.1415 | ZBP1 | 0.1058 |
| TNFSF13B | 0.1365 | CCR1 | 0.1048 |
| IL7R | 0.1332 | CDKN1A | 0.0962 |
| CCL8 | −0.1327 | DYRK2 | −0.0959 |
| SRC | −0.1300 | IL2 | 0.0903 |
| CD44 | −0.1234 | CLEC7A | −0.0890 |
| ARL14EP | 0.1176 | CX3CR1 | 0.0823 |
| STING | −0.1162 | ARL14EP | 0.0783 |
| CCL4 | −0.1116 | IL7R | −0.0770 |
| CCNB1IP1 | 0.1049 | STAT2 | 0.0715 |
| CLEC7A | 0.1015 | IL1A | 0.0574 |
| EIF2AK4 | 0.0968 | IFITM1 | 0.0561 |
| CXCL9 | −0.0958 | CCL2 | 0.0463 |
| C3 | −0.0589 | CCL20 | 0.0461 |
| CD3D | 0.0583 | CD3D | 0.0453 |
| SLAMF7 | −0.0505 | PTGS2 | −0.0444 |
| CCR1 | −0.0371 | EIF2AK4 | 0.0353 |
| TGFB1 | −0.0277 | IFNG | 0.0338 |
| CXCL10 | −0.0211 | TGFB1 | 0.0228 |
| 121601901- | −0.0125 | CD209 | 0.0188 |
| HERV0116 | | | |
| FAM89A | 0.0090 | CCNB1IP1 | 0.0119 |
| ADGRE3 | 0.0008 | CXCL2 | −0.0084 |

Immune Functional Test as a Stratification Tool for Sepsis Patients

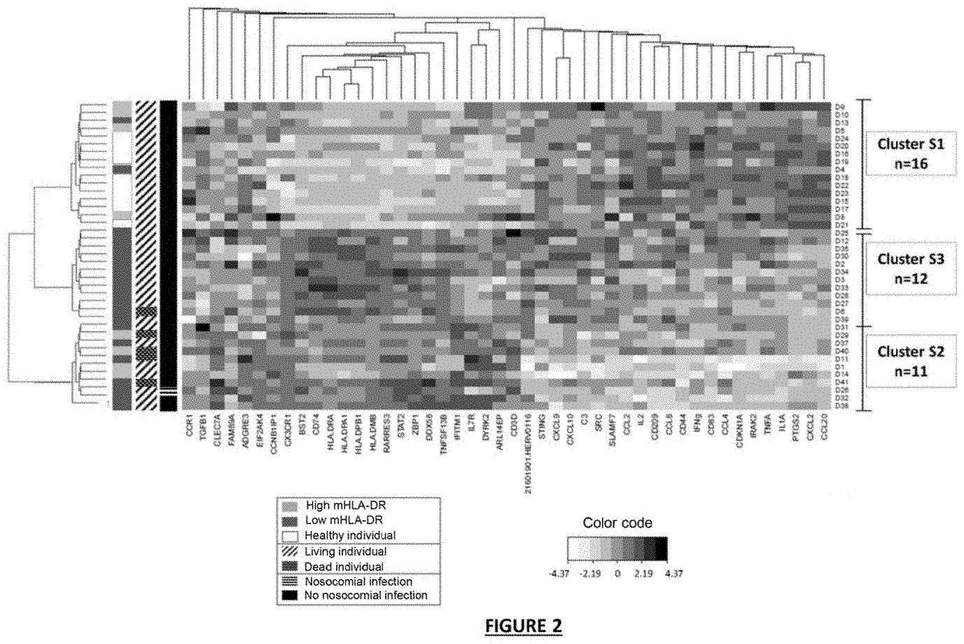

By taking into account the two populations (healthy individuals and patients), we carried out an unsupervised classification (clustering) with the entire molecular panel in order to identify the gene motifs. Healthy individuals were clustered together after SEB stimulation, showing great homogeneity in their immune response. During SEB stimulation, 6 patients were grouped with healthy individuals (n=16, cluster S1) and the others were separated into 2 groups of almost equal number (n=11 for cluster S2 and n=12 for the cluster S3; FIG. 2). The donor composition of each cluster is presented in Table 5.

TABLE 5
Individual composition (per donor) of the clusters obtained after
stimulation with SEB. Healthy individuals appear in italics,
non-survivors in bold, and those having developed
a nosocomial infection are underlined. D: Donor

| Cluster S1 | Cluster S2 | Cluster S3 |
|---|---|---|
| D4; D5; D8; D9; D10; | D1; D11; D14; D26; | D2; D3; D6; D12; D25; |
| D13; D15; D16; D17; | D29; D31; D32; D37; | D27; D28; D30; D33; |
| D18; D19; D20; D21; | D38 ;D40; D41 | D34; D35; D39 |
| D22; D23; D24 | | |

A bivariate analysis was then carried out between the clusters and the biological or clinical parameters.

For SEB stimulation, a statistically significant result was found for mHLA-DR (adjusted p=0.0131) as well as for TNFα protein secretion after LPS stimulation (adjusted p≤0.0001; Table 6).

As expected due to the classification with healthy individuals, the 6 patients in cluster S1 present the highest median for mHLA-DR (10938 Ab/C, IQR:[9456-14642]and present a concentration of highest TNFα protein after LPS stimulation (3799 pg/mL, IQR:[2067.2-5401.2]).

By comparing the results of the clusters S1 and S2, the only significant difference is the median concentration of TNFα protein after stimulation by LPS (p<0.0001). The cluster S2 presents the lowest median TNFα protein level among the 3 clusters.

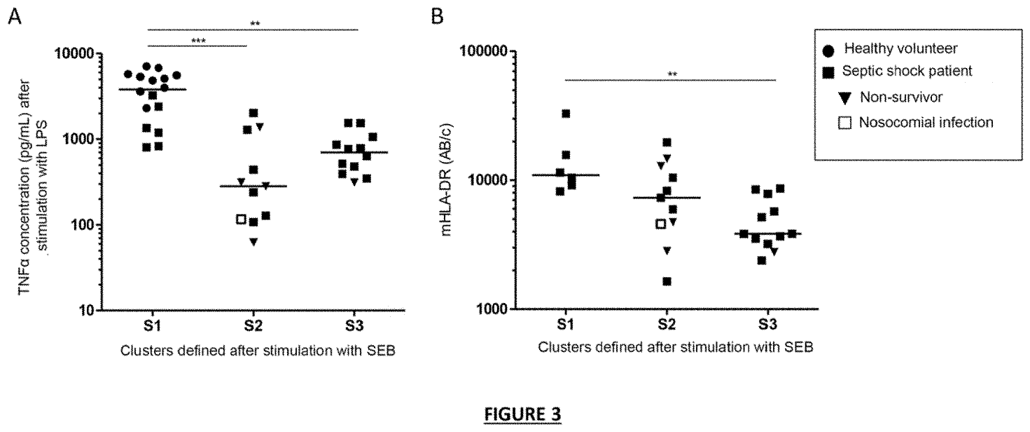

By comparing the cluster S1 to S3, there is a significant difference for the two parameters (p <0.001), the cluster S3 presents an intermediate median level of TNFα protein concentration after stimulation by LPS between the 3 clusters, while the median levels of mHLA-DR are the lowest (FIG. 3).

Moreover, we can observe that among the 20 (out of 30) patients who suffered io from at least one comorbidity, 10 (50%) belonged to S3, representing 83.3% of the cluster.

Similarly, among the 5 non-surviving patients, the four who died before day 28 (80%) belong to cluster S2, representing 36% of this cluster, while the fifth, who died late in hospital, belongs to cluster S3 (Table 6). It should be noted that the only patient who developed a nosocomial infection is in cluster S2.

TABLE 6
Bivariate analyzes between clusters S1, S2 and S3 during SEB
stimulation for clinical and biological parameters. 6 parameters are represented
when statistical analyzes were performed between clusters S1 (n = 16 or n = 6 when
there was no information available for healthy individuals), S2 (n = 11) and S3 (n =
12) defined using the PAM method with correlation distance. The p-value adjusted for
multiple tests was output. The presence of comorbidities was affirmative when at
least one comorbidity was present in the patient: chronic lung disease, heart failure,
myocardial infarction, ulcer, diabetes, renal failure or malignant solid tumor.

| | Cluster S1 | | Cluster S2 | | Cluster S3 | | Adjusted |
|---|---|---|---|---|---|---|---|
| | (n = 16) | | (n = 11) | | (n = 12) | | p-value |
| Status | | | | | | | 0.0002 |
| Healthy individual, n(%) | 10 | (62.5) | 0 | (0) | 0 | (0) | |
| Patients, n(%) | 6 | (37.5) | 11 | (100) | 12 | (100) | |
| Comorbidities* | | | | | | | 0.2348 |
| no, n(%) | 1 | (16.7) | 6 | (54.5) | 2 | (16.7) | |
| yes, n(%) | 5 | (83.3) | 5 | (45.5) | 10 | (83.3) | |
| CCI* median, [IQR] | 2 | [1.2-4.2] | 1 | [0-1.5] | 2 | [1-5] | 0.1593 |
| SOFA* median (day 1), [IQR] | 7.5 | [6.2-8] | 8 | [6.5-10.5] | 8.5 | [8-10] | 0.6383 |
| Mortality* | | | | | | | 0.2416 |
| no, n(%) | 6 | (100) | 7 | (63.6) | 11 | (91.7) | |
| yes, n(%) | 0 | (0) | 4 | (36.4) | 1 | (8.3) | |

| | | | | |
|---|---|---|---|---|
| mHLA-DR* median | 10938 | 7301 | 3839.5 | 0.0131 |
| (day 3-4) (Ab/C), [IQR] | [9456-14642] | [4653-11673] | [3444-6250] | |
| Median TNFα secretion, post- | 3799 | 282.7 | 700.8 | 0.0001 |
| stimulation with | [2067.2-5401.2] | [122.2-861.8] | [457.8-913.3] | |
| LPS (pg/mL), [IQR] | | | | |

SOFA: sequential organ failure assessment
CCI: Charlson Comorbidity Index
HLA-DR: human leukocyte antigen DR
Ab/C: antibodies bound per cell
TNFα: tumor necrosis factor alpha
LPS: lipopolysaccharide
IQR: interquartile range
*: parameters measured exclusively for patients in septic shock

The developed immune functional test has therefore made it possible to demonstrate that, if the immune response of healthy individuals is homogeneous, the immune response of patients with septic shock is heterogeneous, and the heterogeneity of the response lies in the adaptive arm of immunity. Patients grouped in cluster S1, with healthy individuals, have a more «normal»/«healthy» immune profile, unlike other patients. A priori, these patients would not require any particular vigilance and a standard of care would be sufficient. Patients in the cluster S2 correspond to «severe» patients, characterized by a high mortality rate. These patients, whose immunity appears to be strongly impaired and who present a greater probability of mortality, could advantageously benefit from more «aggressive» and/or earlier therapeutic interventions. Finally, the third group (patients of the cluster S3) corresponds to patients with an intermediate to severe phenotype, who may show a degree of immune recovery. Thus, these patients whose immunity seems to be recoverable could be the subject of personalized treatments (e.g. IL-7, interferon γ). Thus, these results show that the immune functional test developed in the context of this invention makes it possible to obtain a stratification of patients, which the reference markers (or gold standard) commonly accepted by the scientific community, such as mHLA- DR or even TNF-α.

1. An in vitro or ex vivo method for determining an individual ability to respond to a stimulus, comprising:
a) A step of incubating a blood sample of said individual with said stimulus, and
b) A step of measuring the expression, from the stimulated blood sample resulting from step a), of at least two different biomarkers, selected respectively from at least two different lists, from the following lists:
List S1: BST2, CCL20, CCL4, CCL8, CD209, CD3D, CD44, CD74, CD83, CLEC7A, CXCL10, CXCL2, CXCL9, DYRK2, FAM89A, HLA-DMB, HLA-DPB1, IFNG, IL1A, IRAK2, PTGS2, RARRES3, DDX58, SLAMF7, SRC, STAT2, STING, TNFA, TNFSF13B, ZBP1;
List S2: ADGRE3, ARL14EP, BST2, C3, CCL2, CCL20, CCL8, CCNB1IP1, IL7R, CD209, CD3D, CD44, CD74, CD83, CDKN1A, CLEC7A, CX3CR1, CXCL10, CXCL2, CXCL9, DYRK2, FAM89A, HLA-DMB, HLA-DPB1, HLA-DRA, IFITM1, IRAK2, SLAMF7, TGFB1;
List S3: 121601901-HERV0116, BST2, C3, CCL20, CCL4, CCL8, CCR1, IL7R, CD209, CD44, CD74, CD83, CLEC7A, CXCL10, CXCL9, EIF2AK4, HLA-DMB, HLA-DPA1, HLA-DPB1, HLA-DRA, IL1A, IL2, RARRES3, SLAMF7, STAT2.

2. The method according to claim 1, wherein the at least two different biomarkers are selected respectively from at least two different lists, from the following lists:
List S1-1: BST2, CCL20, CCL4, CCL8, CD209, CD3D, CD44, CD83, CXCL2, DYRK2, HLA-DMB, IFNG, IL1A, IRAK2, PTGS2, RARRES3, DDX58, SRC, STAT2, STING, TNFA, TNFSF13B, ZBP1;
List S2-1: ADGRE3, ARL14EP, C3, CCL2, CCNB1IP1, IL7R, CD3D, CD44, CDKN1A, CLEC7A, CX3CR1, CXCL2, DYRK2, HLA-DMB, HLA-DRA, IFITM1, IRAK2, TGFB1;
List S3-1: 121601901-HERV0116, C3, CCR1, IL7R, CD44, CD74, CXCL10, CXCL9, EIF2AK4, HLA-DMB, HLA-DPA1, HLA-DPB1, HLA-DRA, IL1A, IL2, RARRES3, SLAMF7, STAT2.

3. The method according to claim 1, wherein the at least two different biomarkers are selected respectively from at least two different lists, from the following lists:
- List S1-2: CCL20, CCL4, CCL8, CD209, CD44, CD83, CXCL2, IFNG, IL1A, IRAK2, PTGS2, DDX58, SRC, STING, TNFA, TNFSF13B, ZBP1;
- List S2-2: ADGRE3, ARL14EP, CCL2, CCNB1IP1, IL7R, CDKN1A, CLEC7A, CX3CR1, DYRK2, IFITM1, TGFB1;
List S3-2: 121601901-HERV0116, C3, CCR1, CXCL10, CXCL9, EIF2AK4, HLA-DMB, HLA-DPA1, IL2, SLAMF7.

4. The method according to claim 1, wherein the at least two different biomarkers are selected respectively from at least two different lists, from the following lists:
List S1-3: IFNG, PTGS2, DDX58, SRC, STING, TNFA, TNFSF13B, ZBP1;
List S2-3: ADGRE3, ARL14EP, CCL2, CNB1IP1, CDKN1A, CX3CR1, IFITM1, TGFB1;
List S3-3: 121601901-HERV0116, CCR1, EIF2AK4, HLA-DPA1, IL2.

5. The method according to claim 1, wherein, in step b), the expression of at least three different biomarkers is measured, selected respectively from each of the three lists.

6. The method according to claim 1, wherein the individual is a patient in a resuscitation unit, in an intensive care unit or in a continuous care unit having received surgery or in a septic state.

7. The method according to claim 1, wherein the blood sample is a whole blood sample.

8. The method according to claim 1, wherein the stimulus comprises a molecule capable of binding at least one type of antigen-presenting cell (APC) and at least one type of adaptive immunity cell.

9. The method according to claim 1, wherein the stimulus comprises a molecule of the superantigen type, selected from the superantigens produced by staphylococcal species and the superantigens produced by streptococcal species.

10. The method according to claim 1, wherein the stimulus comprises a molecule selected from SEB (Staphylococcal Enterotoxin B) and SEA (Staphylococcal Enterotoxin A).

11. The method according to claim 1, wherein the stimulus comprises a molecule analogous to a superantigen, said molecule analogous to a superantigen being a bispecific antibody.

12. The method according to claim 1, wherein the stimulus allows direct activation of T lymphocytes.

13. The method according to claim 1, wherein the stimulus is selected from antibodies recognizing and activating a receptor on the surface of the T lymphocyte.

14. The method according to claim 1, wherein the stimulus is an anti-CD3 antibody.

15. The method according to claim 1, wherein the stimulus is of the imidazoquinoline type.

16. The method according to claim 1, wherein the stimulus is Resiquimod (R848).

17. The method according to claim 1, wherein the stimulus comprises a molecule for therapeutic purposes.

18. The method according to claim 1, wherein the expression of the biomarkers is measured at the messenger RNA (mRNA) level.

19. The method according to claim 1, wherein the expression of the biomarkers is measured by RT-PCR.

20. The method according to claim 1, wherein the expression of the biomarkers is measured by sequencing.

21. The method according to claim 1, wherein the expression of the biomarkers is measured by hybridization.

22. The method according to claim 1, wherein the expression of the biomarkers is normalized with respect to the expression of one or more housekeeping genes.

23. The method according to claim 1, wherein it comprises a step of measuring the expression, from a control blood sample without stimulation, of the same biomarkers as those measured from the stimulated blood sample.

24. The method according to claim 23, wherein it comprises a step of calculating the ratios of the expression of each biomarker in the stimulated blood sample, relative to the expression.

25. A kit comprising means for amplifying and/or detecting at least two different biomarkers, selected respectively from at least two different lists among the lists of claim 1, wherein all of the means for amplifying and/or detecting said kit allow the detection and/or amplification of at most 100 biomarkers, in total.

26. The kit according to claim 25, comprising means for amplifying and/or detecting one or more housekeeping genes.

27. The kit according to claim 25, comprising positive control means making it possible to qualify the quality of the RNA extraction, the quality of any amplification and/or hybridization method.

28. A use of:
means for amplifying and/or detecting at least two different biomarkers, selected respectively from at least two different lists among the lists of claim 1, or
a kit comprising such amplification and/or detection means and optionally, said kit comprises means for amplifying and/or detecting one or more housekeeping genes and/or positive control means making it possible to qualify the quality of the extraction of the RNA, the quality of any amplification and/or hybridization method,
to determine an individual ability to respond to a stimulus.
